(19) United States
(12) Patent Application Publication
(10) Pub. No.: US 2015/0031877 A1
(43) Pub. Date: Jan. 29, 2015

(54) CRYSTAL OF FLUMIOXAZIN

(71) Applicant: SUMITOMO CHEMICAL COMPANY, LIMITED, Tokyo (JP)

(72) Inventor: Mitsunori Hiratsuka, Niihama-shi (JP)

(21) Appl. No.: 14/374,141

(22) PCT Filed: Feb. 8, 2013
(86) PCT No.: PCT/JP2013/053780

§ 371 Date: Jul. 23, 2014

(30) Foreign Application Priority Data
Feb. 16, 2012 (JP) 2012-031376

(51) Int. Cl. C07D 413/04; A01N 43/84

(52) U.S. Cl. CPC C07D 413/04; A01N 43/84
USPC 544/105

(57) ABSTRACT

A crystal of flumioxazin, which shows a powder X-Ray diffraction pattern having diffraction peaks with 2θ values (°) shown in Table.

TABLE
2θ value (°)
9.8 ± 0.1
11.4 ± 0.1
12.7 ± 0.1
13.8 ± 0.1
16.0 ± 0.1
16.4 ± 0.1
16.7 ± 0.1

### TECHNICAL FIELD

The present invention relates to a crystal of flumioxazin.

### BACKGROUND ART

Flumioxazin is sold as a herbicide in many countries, including Japan (Sumitomo Chemical 2001-I, p. 14-15, The Pesticide Manual, 13th ed., British Crop Protection Council, p. 461-462 (2003)). Flumioxazin is a yellowish brown powder solid (Sumitomo Chemical 2001-I, p. 14-25). JP 61-76486 A and JP 5-97848 A mention a method for producing flumioxazin.

### DISCLOSURE OF THE INVENTION

The present invention includes the followings.

[1] A crystal of flumioxazin,
which shows a powder X-Ray diffraction pattern having diffraction peaks with 2θ values (°) shown in Table.

TABLE
2θ value (°)
9.8 ± 0.1
11.4 ± 0.1
12.7 ± 0.1
13.8 ± 0.1
16.0 ± 0.1
16.4 ± 0.1
16.7 ± 0.1

[2] The crystal according to claim [1],
which is an isolated crystal.
[3] A formulation which comprises the crystal according to [1] or [2] as an active ingredient.
[4] An herbicide which comprises the formulation according to [3].
[5] A method for producing an herbicide, which comprises the step of formulating the crystal according to [1] or [2] as an active ingredient to obtain the herbicide.

### MODE FOR CARRYING OUT THE INVENTION

The present invention will be described in detail below.

The crystal of the present invention is an A-type crystal. The A-type crystal shows a powder X-Ray diffraction pattern which has diffraction peaks with 2θ values (°) shown in Table as mentioned above, for example the pattern as follow.

TABLE 1

| 2θ value (°) | d value (Å) |
|---|---|
| 9.8 ± 0.1 | 9.0179 |
| 11.4 ± 0.1 | 7.7556 |
| 12.7 ± 0.1 | 6.9645 |
| 13.8 ± 0.1 | 6.4117 |
| 16.0 ± 0.1 | 5.5347 |
| 16.4 ± 0.1 | 5.4006 |
| 16.7 ± 0.1 | 5.3042 |

The crystal of the present invention can be produced by the methods disclosed in Example and modified methods thereof. The crystal of the present invention can be obtained by dissolving a starting material in an organic solvent to obtain a solution which contains flumioxazine at the concentration in the range of 100 mg to 200 mg per ml of the solvent, and setting the temperature of the obtained solution within the range of 10° C. to 95° C., followed by cooling the solvent to its temperature from about 0° C. to less than 10° C., preferably from about 0° C. to 5° C., to isolate crystals of flumioxazine.

It is possible to use, as the starting material for producing the crystal of the present invention, a solution or a suspension of flumioxazin, or a mixture containing flumioxazin. It is also possible to use a solution or a suspension of a synthetic reaction crude product containing flumioxazin.

It is also possible to use seed crystals in crystallization for producing the crystal of the present invention. In that case, it is preferred to use crystals having a crystal form to be prepared. The amount of seed crystals to be added is preferably from 0.0005 parts by weight to 0.02 parts by weight, and more preferably from 0.001 part by weight to 0.01 part by weight, based on 1 part by weight of flumioxazin.

The crystal of the present invention can be isolated by a filtration, centrifugation, or gradient method. The crystal may be washed with an appropriate solvent, if necessary. Purity and quality of the crystal can be improved by recrystallization or slurry purification.

The crystals of the present invention may be of a solvate or a non-solvate.

When a specific hydrophilic organic solvent is used as a crystallization solvent, the obtained crystals are sometimes crystals of a solvate. The crystals of a non-solvate can be obtained by heating to dry the crystals of a solvate under reduced pressure.

The degree of drying of the crystals can be determined by analytical means such as gas chromatography.

It is also possible to determine the purity of the crystal form of the crystal by subjecting the crystal to the powder X-ray diffraction measurement such as CuKα rays diffraction analysis, followed by analyzing the obtained diffraction pattern about the presence or absence of diffraction peaks peculiar to crystal of a solvate, and the height of the peaks.

The crystal of the present invention can be produced with high purity, can remain unchanged in crystal form even after a heat treating step for formulation, can also exhibit physical and chemical properties which are more advantageous for the production of a formulation, and can maintain such properties even after being stored for a long period.

The crystal of the present invention can be formulated by a method described hereinafter. The formulation which comprises the crystal as an active ingredient is one aspect of the present invention. An herbicide can be obtained by formulating the crystal of the present invention as an active ingredient. The herbicide which comprises the crystal of the present invention, and a method for producing such herbicide fall within the scope of the present application.

When the formulation is prepared from the crystal of the present invention, the crystal are usually mixed with a solid carrier, a liquid carrier, a surfactant, and other auxiliaries for formulation, and then the mixture is formulated into an emulsifiable concentrate, a wettable powder, a suspension concentrate, or a granule. The formulation of the present invention comprises, as an active ingredient, the crystal of the present invention in the amount of 0.05% to 90%, and preferably 0.1% to 80% by weight of the total amount thereof.

Examples of the solid carrier include fine powders or granules of minerals, such as kaolin clay, attapulgite clay, bentonite, acidic white clay, pyrophylite, talc, diatomaceous earth, calcite, walnut shell flour, urea, ammonium sulfate, and synthetic hydrated silicon oxide. Examples of the liquid carrier include aromatic hydrocarbons such as xylene and methylnaphthalene; alcohols such as isopropanol, ethylene glycol, and cellosolve; ketones such as acetone, cyclohexanone, and isophorone; vegetable oils such as soybean oil and cottonseed oil; dimethyl sulfoxide, N,N-dimethylformamide, acetonitrile, and water.

Examples of the surfactant to be used for emulsification, dispersion, and wetting include anionic surfactants such as alkylsulfate ester salts, alkylarylsulfonates, dialkylsulfosuccinates, and polyoxyethylenealkylaryletherphosphate ester salts; and nonionic surfactants such as polyoxyethylenealkylethers, polyoxyethylenealkylarylethers, polyoxyethylene polyoxypropylene block copolymers, sorbitan fatty acid esters, and polyoxyethylene sorbitan fatty acid esters. Examples of other auxiliaries for formulation include ligninsulfonates, alginates, polyvinyl alcohol, gum arabic, carboxymethyl cellulose (CMC), and isopropyl acid phosphate (PAP).

The crystal of the present invention can be used, as active ingredients of the herbicide for agricultural lands such as cultivated lands, paddy fields, orchards, grasslands, lawns, and forests, or non-agricultural lands.

The herbicide or formulation of the present invention can be applied to a soil treatment, a foliagre treatment, or a flooding treatment before or after the germination of weeds. Examples of the soil treatment include a soil surface treatment and a soil mixing treatment. Examples of the foliage treatment include, in addition to a treatment by application from above plants, and a local treatment in which only weeds are treated so as not to apply the herbicide to crops.

Further improvement in the herbicidal effect can be expected by using the herbicide in combination with other herbicides. It is also possible to use it in combination with insecticides, acaricides, nematocides, fungicides, plant growth regulators, fertilizers, and soil conditioners.

When the crystals of the present invention are used as active ingredients of the herbicide, the amount thereof varies depending on the weather conditions, type of the formulation, timing of the treatment, method, place, weed to be killed and crop to be obtained and is usually from 0.02 g to 100 g, and preferably from 0.05 g to 50 g, per are of the land, i.e. per 100 m² of the land to be treated. A predetermined amount of the emulsion concentrate, wettable powder or suspension concentrate is usually diluted with 1 to 10 liters, per are, of water containing, if necessary, an auxiliary such as a spreader before the treatment. The granule is usually used directly without dilution.

Examples of the spreader include, in addition to the above-mentioned surfactants, polyoxyethylene resin acids (esters), ligninsulfonates, abietates, dinaphthylmethanedioulfonates, and paraffin.

### EXAMPLES

The present invention will be described in more detail below by way of Examples.

### Example 1

Flumioxazin (100 mg) was dissolved in methylisobutylketone at 60° C. so as to adjust its concentration to 10.1 mg/mL. The solvent was rapidly cooled to 0° C., followed by being left to stand to obtain A-type crystals.

By X'Pert Pro MPD (manufactured by Nederland PANalytical B.V.), a powder X-ray diffraction pattern of the obtained crystals was measured for each crystal at a scanning range from 2.0° to 40.0° (2θ) using CuKα rays (40 kV, 30 mA).

The pattern of the obtained crystals had the peaks with as 2θ values as shown in Table 2.

TABLE 2

| 2θ value (°) | d value (Å) | Relative intensity (%) |
|---|---|---|
| 9.8 | 9.0179 | 61.1 |
| 11.4 | 7.7556 | 13.1 |
| 12.7 | 6.9645 | 100.0 |
| 13.8 | 6.4117 | 24.1 |
| 16.0 | 5.5347 | 37.9 |
| 16.4 | 5.4006 | 32.4 |
| 16.7 | 5.3042 | 29.1 |

### Formulation Example 1

Fifty (50) parts of the crystals of the present invention, 8 parts of calcium ligninsulfoate, 2 parts of sodium lauryl sulfate, and 45 parts of synthetic hydrated silicon oxide are well ground and mixed to obtain wettable powders.

### Formulation Example 2

Five (5) parts of the crystals of the present invention, 14 parts of polyoxyethylene styryl phenyl ether, 6 parts of calcium dodecylbenzene sulfonate, 80 parts of xylene, and 45 parts of isophorone are well mixed to obtain emulsifiable concentrates.

### Formulation Example 3

Two (2) parts of the crystals of the present invention, 1 part of synthetic hydrated silicon oxide, 2 parts of calcium ligninsulfoate, 30 parts of bentonite, and 65 parts of kaolin clay are well ground and mixed. After adding water, the mixture is well kneaded, and then the kneaded mixture is granulated and dried to obtain granules.

### Formulation Example 4

Twenty-five (25) parts of the crystals of the present invention, 8 parts of polyoxyethylene sorbitan monoleate, 8 parts of CMC, and 69 parts of water are mixed, and then the mixture is wet-ground until the particle size becomes 5 microns or less to obtain suspension concentrates.

### Formulation Example 5

Five (5) parts of the crystals of the present invention, 14 parts of polyoxyethylene styryl phenyl ether, 6 parts of calcium dodecylbenzene sulfonate, 80 parts of xylene, and 45 parts of N,N-dimethylformamide are well mixed to obtain emulsifiable concentrates.

### INDUSTRIAL APPLICABILITY

According to the present invention, crystals of flumioxazin having excellent physicochemical properties can be provided.

1. A crystal of flumioxazin,
which shows a powder X-Ray diffraction pattern having diffraction peaks with 2θ values (°) shown in Table.
TABLE
2θ value (°)
9.8 ± 0.1
11.4 ± 0.1
12.7 ± 0.1
13.8 ± 0.1
16.0 ± 0.1
16.4 ± 0.1
16.7 ± 0.1

2. The crystal according to claim 1,
which is an isolated crystal.

3. A formulation which comprises the crystal according to claim 1 as an active ingredient.

4. An herbicide which comprises the formulation according to claim 3.

5. A method for producing an herbicide, which comprises the step of formulating the crystal according to claim 1 as an active ingredient to obtain the herbicide.
